(19) United States
(12) Patent Application Publication
(10) Pub. No.: US 2026/0188484 A1
(43) Pub. Date: Jul. 2, 2026

(54) SCAN-LESS AUTOMATED PROGRAMMING OF INFUSION DEVICES

(71) Applicant: CareFusion 303, Inc., San Diego, CA (US)

(72) Inventor: Michael K. WORKMAN, Calrsbad, CA (US)

(21) Appl. No.: 19/130,336

(22) PCT Filed: Nov. 15, 2022
(86) PCT No.: PCT/US2022/049997

§ 371 Date: May 15, 2025

(51) Int. Cl. G16H 40/67; G16H 40/40

(52) U.S. Cl. CPC G16H 40/67; G16H 40/40

(57) ABSTRACT

A system for automatically programming an infusion device is disclosed. An infusion device can be activated to send a first message indicating that the medical device is ready to receive automated programming. A terminal, separate from the infusion device, may be used by a user to request an automated programming message be sent to an unidentified medical device, and the terminal displays the medical device as being ready to receive automated programming. The user may then confirm, using the terminal, that the automated programming message is for the medical device and the system automatically causes, responsive to receiving the confirmation, the automated programming message to be transmitted to the medical device, the automated programming message causing the medical device to be configured according to predetermined operational parameters.

### BACKGROUND

This application relates generally to ensuring that an infusion device is properly programmed.

Modern infusion devices provide, via a user interface, manual workflows with regard to programming the pumps for medication infusions. Also, an infusion device may receive infusion order parameters from a third-party system to configure the device's pump to deliver a specific fluid, at specific rates, for a specific patient; a remote programming command often referred to as an automated programming request (APR). The APR configuration may include pre-populating operating parameter values for presentment via the user interface.

Pre-population of infusion parameters reduces the number of programming screens, key presses, and potential input errors that may exist with manual programming. Generally, there may be no differences in programming screens, prompts or the user interface between systems configured for APR and those that are not, and the implementation of APRs does not preclude a clinician from manually programming the pump. However, manual programming is required in the event of a failure in any component of the interfaced system.

APR enabled systems require pump identification which typically is accomplished via entry of the pump identifier into a terminal separate from the pump. A barcode scanner may be connected to the terminal and used to scan the pump's identification from a barcode. When the scanner is broken such entry is typically by the clinician manually keying in the identifier from a keyboard. The process takes time and is prone to errors.

### SUMMARY

The subject technology provides a mechanism and corresponding system for automatically identifying infusion devices ready to receive automated programming. Unlike legacy systems and processes, the subject technology allows the clinician user to publish messages from an infusion device to an APR system, which may then initiate an automated programming message, automatically or on receiving confirmation from the clinician via a terminal. In this manner, the pump is promptly and accurately programmed with the risk of errors in the programming process eliminated.

In this regard, the subject technology includes a medical information system for automatically programming a medical device, comprising one or more computing devices configured to: receive, from a medical device remote from the medical information system, a first message indicating that the medical device is ready to receive automated programming; receive, from a terminal remote from the medical device, a request to automatically program an unidentified medical device according to an automated programming message, the request not identifying the medical device; present for display at the terminal, based on the first message and responsive to the request, the medical device as being ready to receive automated programming; receive, from the terminal after presenting the medical device as being ready to receive automated programming, a confirmation that the automated programming message is for the medical device; and automatically cause, responsive to receiving the confirmation, the automated programming message to be transmitted to the medical device, the automated programming message causing the medical device to be configured according to predetermined operational parameters. Other aspects include corresponding devices, methods, and computer program products for implementation of the corresponding system and its features.

The subject technology also relates to an infusion device, comprising: a pump controller comprising a processor; wherein the pump controller is configured to: determine that the infusion device is in an operational state to program the infusion device to perform a therapy; activate a user interface element configured to transmit, via a communication network, a first message indicating that the infusion device is ready to receive automated programming; receive an activation of the user interface element; responsive to receiving the activation, sending the first message to a medical information system, indicating that the infusion device is ready to receive automated programming; receive, from the medical information system after sending the first message, an automated programming message; and configure, responsive to receiving the automated programming message, the infusion device according to predetermined operational parameters associated with the received automated programming message. Other aspects include corresponding systems, processes, methods, and computer program products for implementation of the corresponding infusion device and its features.

The subject technology also relates to a method for automatically programming a medical device, the method comprising: receiving, from a medical device remote from the medical information system, a first message indicating that the medical device is ready to receive automated programming; receiving, from a terminal remote from the medical device, a request to automatically program an unidentified medical device according to an automated programming message, the request not identifying the medical device; presenting for display at the terminal, based on the first message and responsive to the request, the medical device as being ready to receive automated programming; receiving, from the terminal after presenting the medical device as being ready to receive automated programming, a confirmation that the automated programming message is for the medical device; and automatically causing, responsive to receiving the confirmation, the automated programming message to be transmitted to the medical device, the automated programming message causing the medical device to be configured according to predetermined operational parameters. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example patient care system that includes an infusion device.

FIG. 1B depicts a closer view of a portion of the patient care system shown in FIG. 1A.

FIG. 1C depicts an example of an institutional patient care system of a healthcare organization, according to aspects of the subject technology.

FIG. 2 depicts an example system for automatically programming a medical device, according to aspects of the subject technology.

FIG. 3A depicts a first example flow diagram for automatically programming a medical device, according to aspects of the subject technology.

FIG. 3B depicts a second example flow diagram for automatically programming a medical device, according to aspects of the subject technology.

FIG. 4 depicts a first example process for automatically programming a medical device, according to aspects of the subject technology.

FIG. 5 depicts a second example process for automatically programming a medical device, according to aspects of the subject technology.

FIG. 6 is a conceptual diagram illustrating an example electronic system for automatically programming a medical device, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Scanning barcodes to identify infusion devices or their connected modules have become problematic, particularly when the barcodes are on the back of the device or have faded over time. The subject technology provides a system for publishing medical devices ready to receive automated programming requests (APRs), commands that cause the device to automatically configure itself with operational parameters to administer a therapy to a patient.

According to various aspects of the subject technology, instead of scanning a barcode or manually entering an identifier for a medical device, the medical device can be activated to send a message to a central server indicating that the medical device is ready to receive automated programming. A terminal, separate from the infusion device, may be used by a user to request an automated programming message/command be sent to the medical device without identifying the medical device. In other words, the user may request an APR be sent to an unidentified medical device not yet known.

The terminal publishes which medical devices (e.g., in the care area of the user) are ready to receive automated programming. The user may then confirm by selection, using the terminal, that the automated programming command currently requested is for the medical device and the system automatically causes, responsive to receiving the confirmation, the automated programming command to be transmitted to the medical device. In this manner, the automated programming command causes the medical device to be configured according to predetermined operational parameters.

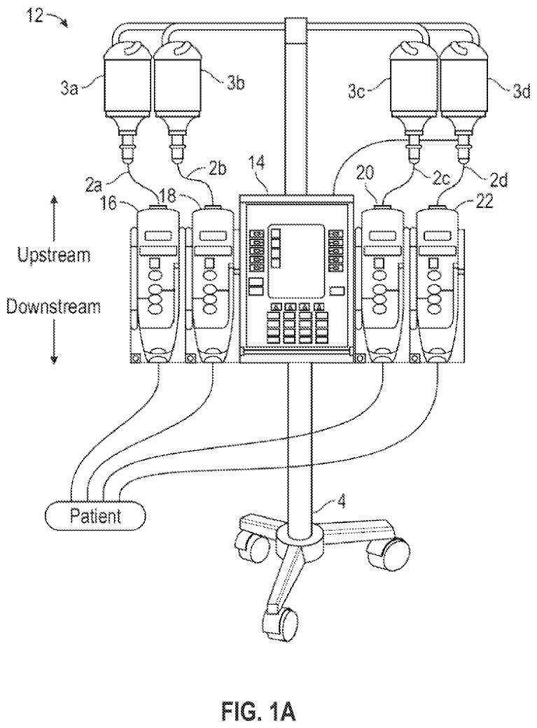

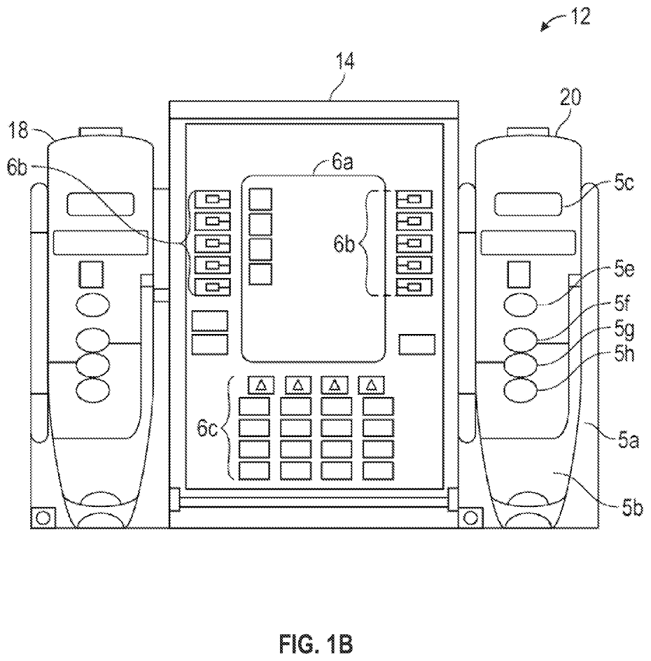

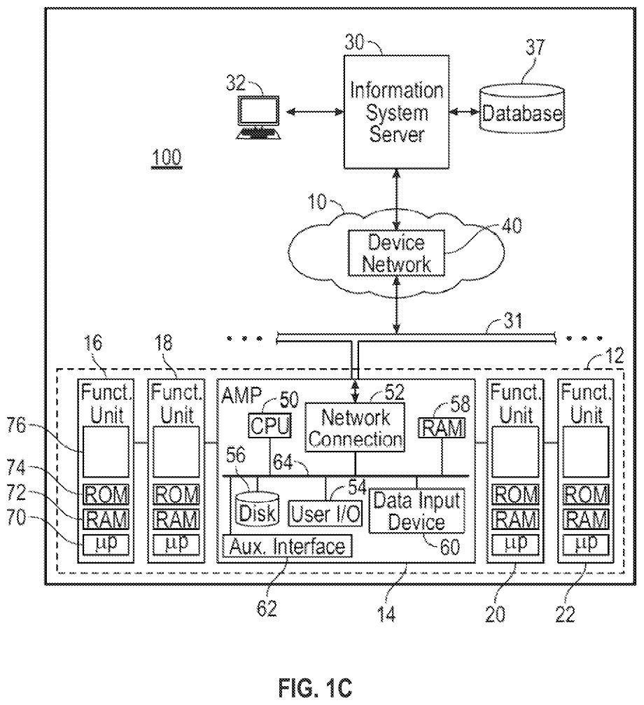

FIG. 1A is an example patient care system, according to various aspects of the subject technology. The patient care system 1 shown in FIG. 1A includes four fluid infusion pumps 16, 18, 20, 22, each of which is in operative engagement with a respective fluid administration set 2a-d. Fluid supplies 3a-d, which may take various forms but in this case are shown as bottles, are inverted and suspended above the pumps. Fluid supplies may also take the form of bags or other types of containers. Both the patient care system 1 and the fluid supplies 3a-d are mounted to a roller stand or pole 4. The specific fluid supplies as well as their orientation (e.g., mount location, mount height, mounting type, etc.) within the care area may generate one or more interaction records. The interaction record for a set for example may be generated in part by detecting a scannable code associated with the set or detecting a physical structure on the set that encodes identifying information for the set prior to use.

As shown in the example implementation of FIG. 1A, each administration set 2a-d is connected between a respective fluid supply 3a-d and the same patient so that the patient may receive the fluids in all the fluid supplies. The administration set may be identified either actively by, for example, scanning by a clinician or passively by, for example, wireless or optical detection of the administration set.

In the depicted example, a separate infusion pump 16, 18, 20, 22 is used to infuse each of the fluids of the fluid supplies into the patient. The infusion pumps are flow control devices that will act on the respective tube or fluid conduit of the fluid administration set to move the fluid from the fluid supply through the conduit to the patient. Because individual pumps are used, each may be individually set to the pumping or operating parameters required for infusing the particular medical fluid from the respective fluid supply into the patient at the particular rate prescribed for that fluid by the clinician.

Typically, medical fluid administration sets have more parts than are shown in FIG. 1A. Many have check valves, drip chambers, valved ports, connectors, and other devices well known to those skilled in the art. These other devices have not been included in the drawings so as to preserve clarity of illustration.

FIG. 1B is a closer view of a portion of the example patient care system shown in FIG. 1A, according to various aspects of the subject technology. FIG. 1B shows two of the fluid infusion pumps mounted at either side of a programming module, and the displays and control keys of each, with the programming module being capable of programming both infusion pumps. The infusion device 12 includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages). Control keys 5e-h exist for programming and controlling operations of the infusion pump as desired. In some implementations, the control keys may be presented as interactive elements on the display 5c (e.g., touchscreen display). The infusion device 12 and/or infusion pump 20 may also include audio alert equipment in the form of a speaker (not shown).

The programming module 14 of the infusion device 12 includes a display 6a for visually communicating various information, such as the operating parameters of a connected pump and alert indications and alert messages, and control keys 6b and 6c for selecting and/or setting control parameters and/or options for controlling the infusion device 12 and connected modules. The programming module 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The programming module 14 may include a communications system (not shown) with which the programming module 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). The communication module may be used to transfer access and interaction information for clinicians encountering the programming module or device coupled therewith (e.g., pump 20 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the programming module 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

In some embodiments, the pressure measurements from the upstream and/or downstream pressure sensors are transmitted to a server or other coordination device, and the methods disclosed herein are implemented on the server or other coordination device. For example, more sophisticated and computationally intensive approaches like machine-learning can be implemented on the server (or on a PCU with a larger memory and/or CPU resources). In some embodiments, machine learning is used to identify empty conditions based on pressure signals received from the pump.

FIG. 1C depicts an example of an institutional patient care system 100 of a healthcare organization, according to aspects of the subject technology. In FIG. 1C, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1C optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1C to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1C, at least one of functional modules 16, 18, 20, 22 may be an infusion pump module such as an intravenous infusion pump for delivering medication or other fluid to a patient. For the purposes of this discussion, functional module 116 is an infusion pump module. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may be a printer, scanner, bar code reader, near-field communication reader, RFID reader, or any other peripheral input, output or input/output device.

Each functional module 16, 18, 20 and/or 22 communicates directly or indirectly with interface unit 14, with interface unit 14 providing overall monitoring and control of device 12. Functional modules 16, 18, 20 and/or 22 may be connected physically and electronically in serial fashion to one or both ends of interface unit 14 as shown in FIG. 1C, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1C, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1C), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information system (HIS) server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

According to various implementations, server 30 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information system 30. The pharmacy reviews the order, and once the order has been prepared, the order may be transmitted to the nurse station for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The establishment of these parameters, along with parameters for off-formulary orders, via the system 30 is useful for maintaining consistency across the healthcare environment and ensuring an order is intelligible and executed according to expectations by other devices within the system 30 (e.g., an infusion pump).

With further reference to FIG. 1C, patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the interface unit 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

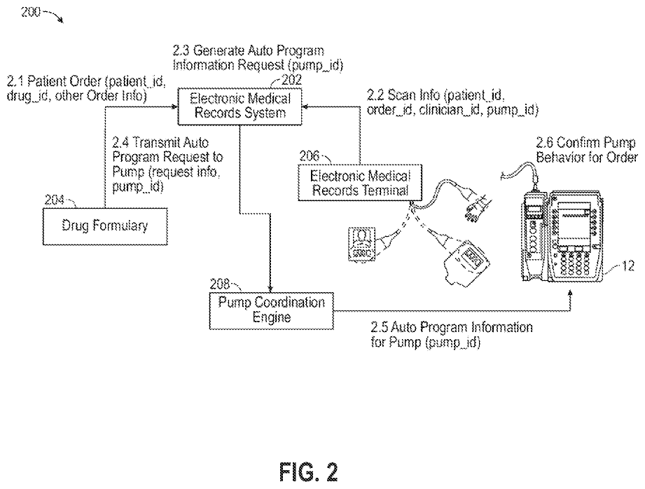

FIG. 2 depicts an example system 200 for automatically programming a medical device, according to aspects of the subject technology. Interoperability between a hospital's electronic medical records (EMR) 202 and medical devices such as infusion device 12 enable pre-population of infusion parameters. Pre-population of infusion parameters may reduce the number of programming screens and key presses required with manual programming of a pump. The implementation of interoperability does not preclude a clinician from manually programming the infusion device. Manual programming may be required in the event of a failure in any component of the interfaced system.

While features may be described with reference to an EMR, the features are applicable to provide scan-less auto-programming of medical devices using similar hospital information systems such as pharmacy data management systems (PDMSs). Furthermore, while the features may be described using an infusion pump as the example medical device, the features are applicable to provide scan-less auto-programming of other medical devices using barcodes for association such as patient monitors, patient association management systems, or alarms management systems.

A formulary 204 determines which medications can be dispensed within a hospital network. A hospital committee may be formed to determine how medications within that formulary would be applied to the patient care devices 12. Configuration definitions (e.g., by hospital unit such as ICU, NICU, Pediatrics, Oncology, Surgery, etc.) are agreed upon and the drugs and typical infusion protocols are established in a medical device drug library (“drug library”). In addition, limitation, or guard rail, conditions are defined in the drug library. When the definitions are complete, then a configuration can be released including the drug library. Pumps at the institution are then updated by transferring the configuration databases into some or all of their pumps. Corresponding updates to the formulary may be shared with other hospital systems such as the pharmacy ordering system or electronic medical records system which may be use formulary information to generate a patient order to deliver a particular drug to a particular patient (e.g., FIG. 2 at 2.1).

In the clinical field, a clinician may scan a medical item such as an infusate package using a scanner associated with a medical device such as an infusion device 12. For example, a bar code reader (or other data input device) is used to scan the coded drug label, the patient's coded ID band and the caregiver's ID badge, and optionally supplementary prescription information or medical device configuration instructions (including configuration database ID) printed on the label or an accompanying order. The reader/scanner is not required to be integrated with a medical device. The scanner may be part of a separate device such as a medical records terminal 206 (e.g., part of one or more computing devices) connected to the same network 40 as the infusion device 12 and configured with software to function in an overall workflow involving the infusion device 12. The scanning initiates a process by which information pertaining to the item (e.g., scanned from a code affixed to or transmitted by the item) is automatically sent (e.g., FIG. 2 at 2.2) to the hospital's EMR 202 (e.g., at a centralized server 30) via a network 40. The EMR 202 may confirm the item and generate (e.g., FIG. 2 at 2.3) and send an automated programming request (APR) to the medical device 12 to load parameters pertaining to the item. The parameters may be stored in the medical device 12, but loaded in response to an identifier received from the server. While the examples herein involve an infusion device, any medical device may be configured in the same or similar manner and employ the automated programming error mitigation described herein.

In the depicted implementation, a coordination engine 208 coordinates messages sent from the EMR 202 to the infusion device 12. In the depicted implementation, the EMR 202 transmits an APR (e.g., FIG. 2 at 2.4) to the pump coordination engine 208 with a device identifier (ID) of the infusion device to receive the APR. The coordination engine then determines whether the infusion device 12 identified by the EMR is available and, if so, forwards the APR to the device 12 (e.g., FIG. 2 at 2.5). When an APR received by an infusion device 12, the infusion device 12 programs itself according to the parameters of the APR. In some implementations, the APR activates a drug library stored on the device, and the infusion device is programmed according to parameters stored in the drug library for the medication identified in the APR. In some implementations, upon successful programming, the infusion device may automatically initiate operation based on the parameters. In some implementations, the infusion device may confirm the automatically entered parameters (e.g., FIG. 2 at 2.6). The confirmation may include presenting one or more user interface screens including the parameters and values along with a control element (e.g., a button) that, when activated, causes the infusion device to begin operation based on the parameters. The user interface may include additional or alternative control elements to allow a clinician to adjust an automatically entered parameter based on, for example, professional judgement or changes in patient condition.

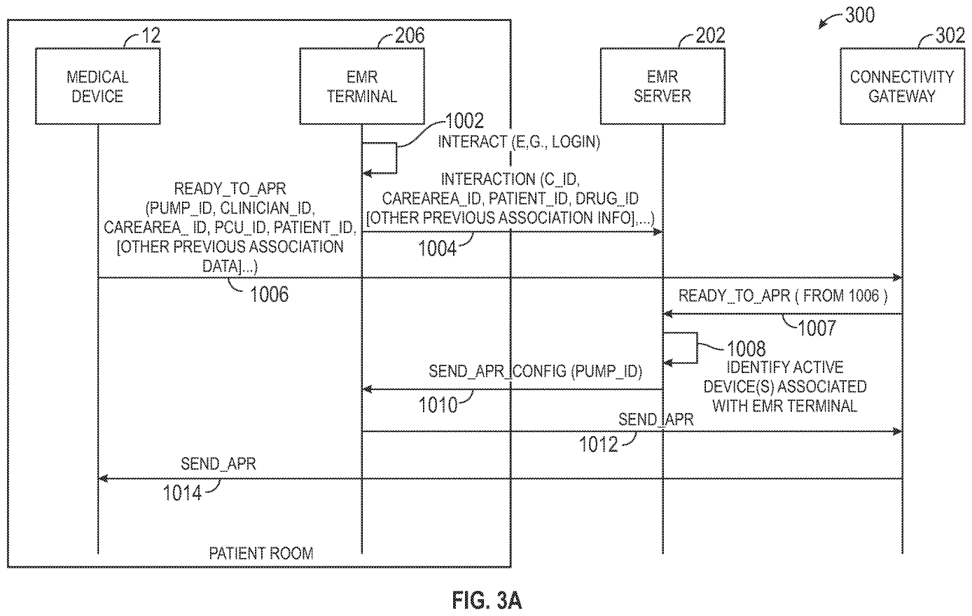

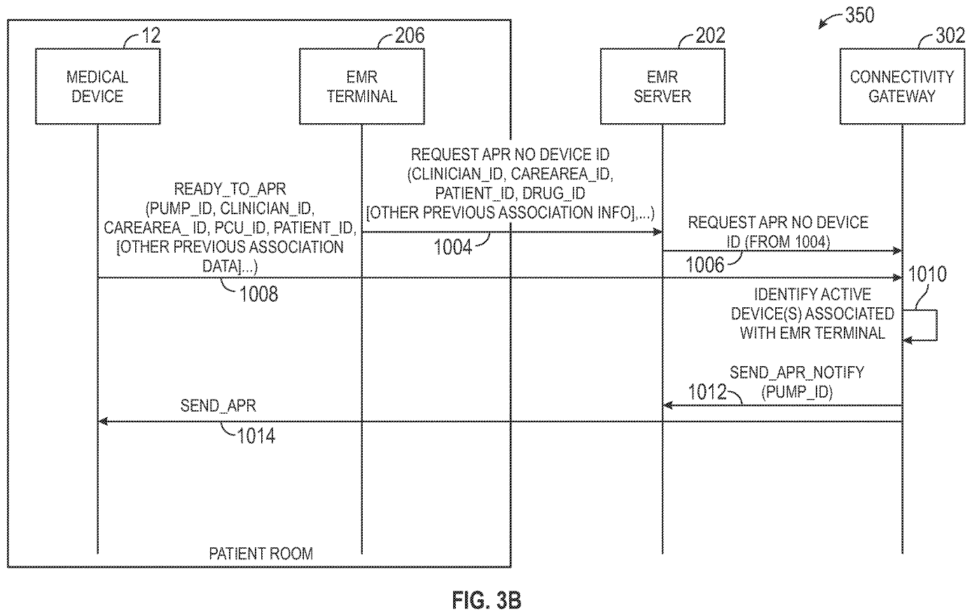

FIG. 3A depicts a first example flow diagram 300 for automatically programming a medical device, according to aspects of the subject technology. For explanatory purposes, the various blocks of example process flow 300 are described herein with reference to FIGS. 1A, 1B, 1C, and 2, and the associated components and/or processes described herein.

As described previously, an identifier of a medical device 12 may be scanned at an EMR terminal 206 and a process is initiated by which information pertaining to the medical device is automatically sent to the hospital's EMR 202 and the EMR 202 performs certain actions pertaining to the medical device and generates and sends an automated programming request (APR) to the medical device 12 to configure the device 12. In some implementations, an identifier of the device 12 is scanned from a barcode affixed to the device in conjunction with parameters entered into the terminal for selecting and generating the APR.

The subject technology provides a mechanism to automatically program a medical device without a scanner or manual translation of identifiers between the medical device and a terminal such as EMR terminal 206. The scan-less device identification functionality of the subject technology enables a user to explicitly publish message from a medical device to any data consumer (e.g., an EMR system) based on automated device identification propagation. The identifier is automatically sent to the consumer, which then correlates the identifier with other data to generate the APR and send the APR instructions back to the medical device.

Using infusion devices as an example, a hospital system implementing the subject technology may include one or more infusion devices 12, an EMR terminal 206, and an EMR server 202 connected to a connectivity gateway 302. In some implementations, the connectivity gateway 302 is part of or associated with the coordination engine 208. In some implementations, the connectivity gateway 302 is a separate system. In some implementations, the gateway 302 may include one or more computing devices such a server or servers apart from the EMR server 202. In some implementations, the gateway 302 may be implemented by or interchangeable with the coordination engine 208 of FIG. 2. In some implementations, the EMR server and the connectivity gateway may co-exist as a single server or a group of servers. Server 30 of FIG. 1C may be representative of EMR server 202 and/or connectivity gateway 302.

According to various implementations, the infusion pump (12) includes a user interface element that, when activated, is configured to transmit, via a communication network 40, a message indicating that the infusion device is ready to receive automated programming. For example, the infusion device (12) may display on its display screen 6a a virtual button as the interface element. The virtual button may be activated by touch activation in implementations where display screen 6a is a touch screen, or may be activated by selecting a corresponding control key 6b or 6c.

In the depicted example of FIG. 3A, a clinician interacts with and authenticates to the EMR terminal 206 at 1002. As described with regard to FIG. 2, the terminal 206 may obtain the clinicians identification by way of the authentication process, and the clinician may enter the care area, patient identifier, drug identifier, or other identifying information for requesting an APR be sent to an infusion device 12. Via messaging 1004, the identifying information obtained via the terminal 206 is transmitted to the EMR server 202 as a request to automatically program an infusion device, but without an identification of the infusion device to be programmed and the infusion device to program is not yet known by the system.

Separately, via messaging 1006, the interface element is activated and the infusion device 12 (e.g., PCU 14 or a connected module) sends a message to the connectivity gateway 302 indicating that the infusion device is ready to receive automated programming. The message may include, for example, parameters such as an identifier of the device (e.g., a serial number), an identifier of a clinician assigned to the pump (e.g., a clinician logged into the device), a care area of the device, a patient control unit identifier (e.g., if the device is part of a PCU 14, and/or an identifier of a patient associated with the infusion device, and the like. the pump controller is configured to:

In some implementations, the interface element is activated after a determination is made (by the device or a connected hospital information system 30) that the infusion device is in an operational state to be programmed to perform a therapy (e.g., to administer a medication to a patient). The interface element may be a virtual button displayed on a touchscreen 6a, or may include a control key 6b or 6c associated with the option of sending the message indicating that the device is ready to receive automated programming. In some implementations, each active channel on the device 12 (e.g., each module 16, 18, 20, 22) may be associated with a virtual button and/or control key, and the clinician may select the appropriate interface element to send the message on behalf of the channel.

The connectivity gateway 302 informs the EMR server 202 that the infusion device 12 is ready to receive automated programming (“Ready to APR”) via messaging 1007. The EMR server 202, based on the request (e.g., messaging 1004) sent by the EMR terminal 206 searches for and identifies all active medical devices that have indicated they are ready to receive automated programming (e.g., via messaging 1008) and returns a list of the active and ready devices to the EMR terminal (e.g., via messaging 1010). The clinician then finds the infusion device 12 (or module) within the list and selects it for automated programming according to the previously entered request (e.g., via messaging 1004). Once selected, the EMR terminal 206 sends the selected identification of the infusion device to the connectivity gateway 302 via messaging 1012, and the gateway 302 associates the device identifier with the APR and sends the APR to the infusion device such as via messaging 1014.

After the APR has been accepted and processed by the infusion device without failure, the infusion device will start and begin to infuse a medication according to the programming provided by the APR. After the infusion has begun, the infusion device will send one or more status messages to the EMR system 202. A status message informs the EMR system what was started and includes certain values on which the infusion device is reporting. This information may reflect the APR data and/or may include changes made at the device. The EMR system may wait for the status message and, upon receiving the message, closes out the workflow pertaining to the APR.

FIG. 3B depicts a second example flow diagram 350 for automatically programming a medical device, according to aspects of the subject technology. For explanatory purposes, the various blocks of example process flow 300 are described herein with reference to FIGS. 1A, 1B, 1C, 2, and 3A and the associated components and/or processes described herein.

In the depicted second example of FIG. 3B, the request for APR originating at the EMR terminal 206 and the APR is associated with an infusion device (e.g., pump 12) at the connectivity gateway 302 automatically rather than at the EMR server 202 as shown in FIG. 3A. In this regard, EMR terminal 206 may request autoprogramming for a patient or drug order without including a device identifier via messaging 1004. Via messaging 1006, the gateway 302 receives the request from the terminal 206 and, via separate messaging 1008, an indication that the pump 12 is ready to receive an APR. Based at least in part on the information included in messaging 1006 and 1008, at 1010, the connectivity gateway 302 automatically associates the device 12 and the EMR terminal 206. This allows the previously unassociated APR request from the EM terminal 206 to be routed to the device 12. For example, the medical device 12 may provide a clinician identifier, a care area identifier, and/or a patient identifier. Similarly, the clinician may enter the same information at the EMR terminal 206. If these data points correspond then the connectivity gateway 302 may automatically provide the APR without further human interaction. In some implementations the correspondence may be based on time. For example, the gateway 302 may compare messages received from the medical device 12 and the EMR server 202 within one minute, two minutes, three minutes, five minutes, ten minutes, or other period of time of each other. Having received a device identifier with each device ready indication, the gateway 302 notifies the EMR server 202 of the association such as via messaging 1012 and directs the APR (created based on the data entered at the EMR terminal) to the device associated with the matching data such as via messaging 1014.

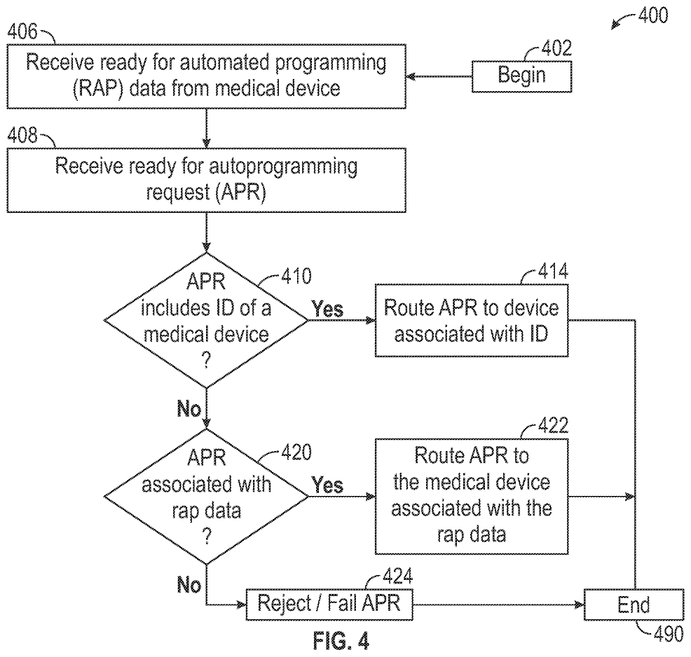

FIG. 4 depicts a first example process 400 for automatically programming a medical device, according to aspects of the subject technology. For explanatory purposes, the various blocks of example process 400 are described herein with reference to FIGS. 1A-C, 2, 3A and 3B, and the associated components and/or processes described herein. The one or more of the blocks of process 400 may be implemented, for example, by one or more computing devices including, for example, EMR server 202 and/or connectivity gateway 302. In some implementations, one or more of the blocks may be implemented based on one or more machine learning algorithms. In some implementations, one or more of the blocks may be implemented apart from other blocks, and by one or more different processors or devices. Further, for explanatory purposes, to the extent that the blocks of example process 400 are described as occurring in serial, or linearly, in some implementations, multiple blocks of example process 400 may occur in parallel (e.g., blocks 406 and 408 may occur in parallel). In addition, the blocks of example process 400 need not be performed in the order shown and/or one or more of the blocks of example process 400 need not be performed.

The process 400 begins (402) by a computing system (e.g., the EMR server and/or connectivity gateway) receiving an indication that a medical device 12 is ready to receive automated programming (RAP) (406). A request to send an automated programming request (APR) is also received from a terminal 206 (or associated EMR server) (408). The process proceeds by determining whether the APR includes an identifier for a medical device (410). If it does then the system may route the APR to the device associated with the identifier (414) and the process ends (490). According to various implementations, the APR received in step 408 does not include an identifier of the medical device 12, and the system proceeds to determine an association between the APR and the medical device 12.

In the example process, the computing system determines whether an APR is associated with a medical device (420). In this regard, the system may compare data in the APR to data received with the RAP indication of step 406, such as care area, patient identifier, caregiver identifier, and the like with information received with the APR request. If the data matches then the system routes the APR to the medical device associated with the RAP data (422). In some implementations, the system may request confirmation from a clinician prior to routing the APR. For example, the system may indicate (e.g., via the terminal 206) that the APR matches a particular device for which a RAP indication was received and prompt the clinician (e.g., logged into the terminal) to confirm the association. In some implementations, multiple RAP indications may have been received during a period of time. In such implementations, the system may provide a list of devices (e.g., for which a RAP was received during a predetermined period) that may be further filtered by the clinician to narrow the list and select the device from the list (e.g., using terminal software interface). If the system cannot find an association between the APR and a device then the system may reject the APR (424).

The rejection or failure at 424 may include transmitting a message to the EMR terminal or server associated with the APR request. The message may, in some instances, include information identifying medical devices having submitted RAP messages but not yet associated with an APR request. In this way, the receiving system can present options via a user interface to receive a selection from devices indicating a readiness for programming. Such request may be submitted in a similar fashion as described in block 408 but including the device identifier associated with the selected device.

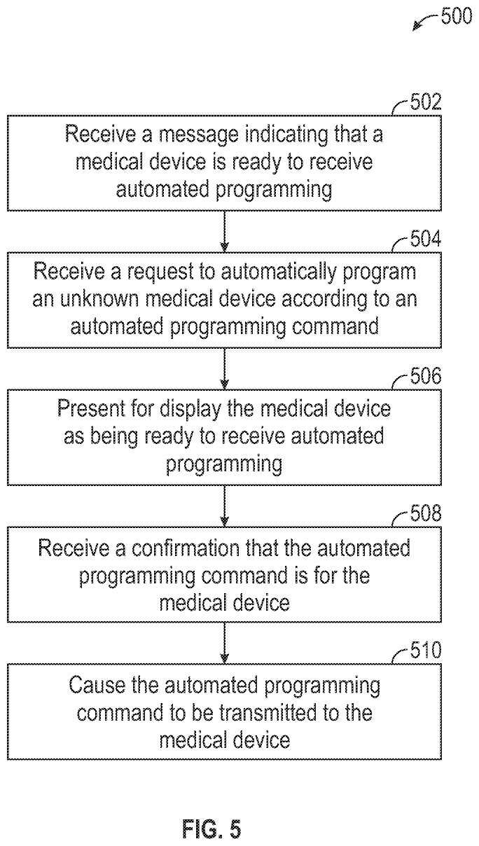

FIG. 5 depicts a second example process 500 for automatically programming a medical device, according to aspects of the subject technology. For explanatory purposes, the various blocks of example process 500 are described herein with reference to FIGS. 1A-C, 2, 3A, 3B, and 4 and the associated components and/or processes described herein. According to various implementations, process 500 is a streamlined variation of process 400. The one or more of the blocks of process 500 may be implemented, for example, by one or more computing devices including, for example, EMR server 202 and/or connectivity gateway 302. In some implementations, one or more of the blocks may be implemented based on one or more machine learning algorithms. In some implementations, one or more of the blocks may be implemented apart from other blocks, and by one or more different processors or devices. Further, for explanatory purposes, to the extent that the blocks of example process 400 are described as occurring in serial, or linearly, in some implementations, multiple blocks of example process 400 may occur in parallel. In addition, the blocks of example process 500 need not be performed in the order shown and/or one or more of the blocks of example process 500 need not be performed.

According to various implementations, a medical information system (e.g., EMR server 202 and/or gateway 302) receives a message indicating that a medical device is ready to receive automated programming (502). In some implementations, the message may be received directly from the medical device remote from the medical information system. In some implementations, the message may be received from another system which obtains identifiers of devices that are active and in a ready state. For example, a server 30 may query medical devices in a hospital network to determine which of the devices have been placed in a ready state, for example by way of a clinician activating a user interface element on the device, and cache device identifiers of active and ready devices.

According to various implementations, the system receives a plurality of messages for a plurality of medical devices (e.g., each from a different medical device) indicating that medical devices are ready to receive automated programming. In a busy hospital, for example, multiple clinicians may be requesting programming for different devices and/or different patients across the hospital organization during a same period of time.

The medical information system also receives a request to automatically program an unknown (e.g., unidentified) medical device according to an automated programming command (504). For the purpose of this disclosure, an automated programming command includes or is part of an APR message sent to a medical device to cause a programming of the medical device. As described previously, a request to send an APR may originate from an EMR terminal 206 and may not identify any particular medical device. A clinician may log into to the EMR terminal 206 at a patient's bedside or at a nurses station and enter certain information for requesting automated programming of a medical device associated with the clinician's patient. In a simple example, the clinician may merely log into the terminal and the terminal may only know the identification of the clinician.

The medical information system presents for display, for example at a terminal 206, the medical device as being ready to receive automated programming (506) (e.g., via an APR). The presentation may appear responsive to the request received at terminal 206. For example, on receiving the request, the system may query a database 37 for all active medical devices 12 currently active and identified as being ready to receive an APR. For example, where multiple devices across the hospital organization have been activated and sent ready requests, all the devices may be returned by the database. In some implementations, the results returned by the query may automatically be filtered based on information received in the request (504). For example, the results may be filtered based on the care area or the clinician who initiated the request.

In some implementations, the medical device may have been previously associated with a care area or a clinician. The care area and/or clinician may also be identified in the message indicating that the device is ready to be programmed, or may be obtained by the medical information system by indexing a database based on the received identifier of the device. The data received from the terminal 206 may then include an identification of the care area by the clinician, or the clinician's identification. The system may then determine a subset of the medical devices that are associated with a care area and/or clinician that match the care area and/or clinician obtained based on the data received from the terminal 206, and present the subset of medical devices as being ready to receive automated programming. The clinician may then select the medical device to receive the APR.

In some implementations, the device listed in the database may be a channel designation for a module 16, 18, 20, 22. In this regard, the system may receive (e.g., in the request(s)) channel designations for a single PCU 14 or for many PCUs across the network. In some implementations, the medical information system may receive an identifier of a PCU and determine the channel designations of modules connected to the PCU by indexing the database based on the identifier of the PCU. Active and/or non active channels may be stored in the database together with their respective status (e.g., active and ready), and the active and ready channels returned by the database as available for selection.

The medical information system may then receive (from the terminal after presenting the medical device as being ready to receive automated programming) a confirmation that the automated programming command is for the medical device (508). For example, the clinician may review the active and ready devices listed at the terminal 206 and select the device that should receive the APR requested by the clinician. The selection may be for a standalone infusion device, or may be for a channel designation corresponding to a patient care unit 14. For example, a clinician may review infusion devices and/or channels in the clinician's care area that are ready to receive automated programming, and make a selection for which of the listed devices to send the APR.

In some implementations, an infusion status of the device may be listed at the terminal. For example, the infusion status may indicate that the infusion device and/or channel is idle, infusing a fluid, infusing medicine (e.g., an acyclovir), busy, etc. The clinician may then review the statuses and determine whether to continue with the selection of the device. If a channel is identified as busy due to an ongoing current infusion, the clinician may elect to have the system send the automated programming command to the device to program an infusion to occur after the current infusion completes.

Responsive to receiving the confirmation, the medical information system causes the automated programming command to be transmitted to the medical device 12 (510). In this regard, the automated programming command causes the medical device to be configured according to predetermined operational parameters. The operational parameters may be stored in a drug library on the device or PCU associated with the device (e.g., associated with the channel). According to various implementations, the automated programming command causes the medical device to be configured by drug library information stored in a drug library deployed to the medical device.

If the medical device to be configured is a module corresponding to a channel designation then the system may send the automated programming command directly to the channel or may send the command to a PCU 14 to which the module is connected and the PCU may transmit the command to the module or program the module based on the command.

Many of the above-described example processes 400, 500, and 700 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

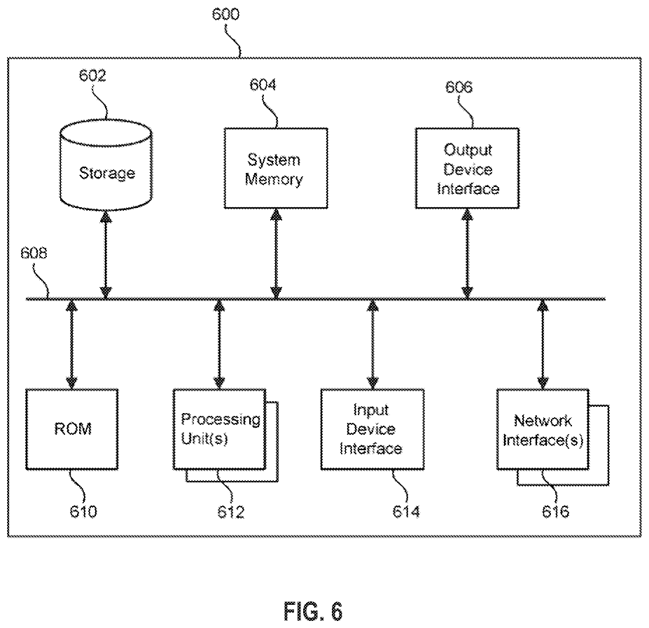

FIG. 6 is a conceptual diagram illustrating an example electronic system 600 for automatically programming a medical device, according to aspects of the subject technology. Electronic system 600 may be a computing device for execution of software associated with one or more portions or steps of processes 300, 350, 400, and 500 or components and methods provided by FIGS. 1-5, including but not limited to computing hardware within server 30, terminal 32, 206, or patient care device 12, and/or any computing devices or associated terminals disclosed herein. In this regard, electronic system 600 may be a specifically configured personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or similar computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry specifically configured for operation of the various components and methods previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as, random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 408 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware or network interface module) for connecting the computer to a part of a network of computers such as a local area network (“LAN”), a wide area network (“WAN”), wireless LAN, or an Intranet, or a network of networks, such as the Internet. Any or all components of electronic system 600 can be specially configured to be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A medical information system, comprising: one or more computing devices configured to: receive, from a medical device remote from the medical information system, a first message indicating that the medical device is ready to receive automated programming; receive, from a terminal remote from the medical device, a request to automatically program an unidentified medical device according to an automated programming command, the request not identifying the medical device; present for display at the terminal, based on the first message and responsive to the request, the medical device as being ready to receive automated programming; receive, from the terminal after presenting the medical device as being ready to receive automated programming, a confirmation that the automated programming message is for the medical device; and automatically cause, responsive to receiving the confirmation, the automated programming message to be transmitted to the medical device, the automated programming message causing the medical device to be configured according to predetermined operational parameters.

Clause 2. The medical information system of Clause 1, wherein the one or more computing devices are further configured to: receive a plurality of messages indicating that a plurality of medical devices are ready to receive automated programming; and present, for display at the terminal, the plurality of medical devices as being ready to receive automated programming, wherein the confirmation is based on a selection at the terminal of the medical device from the plurality of medical devices.

Clause 3. The medical information system of Clause 2, wherein receiving the plurality of messages comprises receiving a plurality of channel designations corresponding to a plurality of medical device modules coupled to a patient care device; wherein presenting the plurality of medical devices as being ready to receive automated programming comprises presenting the plurality of channel designations for the patient care device; wherein the confirmation comprises a selected channel designation of the plurality of channel designations; and wherein the automated programming message instructs the patient care device to configure a medical device module corresponding to the selected channel designation according the predetermined operational parameters.

Clause 4. The medical information system of Clause 3, wherein receiving a plurality of messages indicating that the plurality of medical devices are ready to receive automated programming comprises: receiving an identifier of the patient care device; and determining the plurality of channel designations based on indexing a database based on the identifier of the patient care device.

Clause 5. The medical information system of Clause 3 or Clause 4, wherein the one or more computing devices are further configured to: receive an infusion status associated with each of the plurality of channel designations; and present, for display at the terminal, each infusion status together with each associated channel designation.

Clause 6. The medical information system of any one of Clauses 3 through 5, wherein the automated programming message causing the medical device to be configured according to predetermined operational parameters comprises the automated programming message causing the medical device to be configured by drug library information stored in a drug library deployed to the medical device.

Clause 7. The medical information system of any one of Clauses 2 through 6, wherein the one or more computing devices are further configured to: receive, for each respective medical device of the plurality of medical devices, a care area associated with the respective medical device; receive, from the terminal after presenting the plurality of medical devices, a selected care area; determine a subset of the plurality of medical devices based matching the selected care area with a care area of one or more of the respective medical devices; and present, for display at the terminal, the subset of the plurality of medical devices as being ready to receive automated programming, wherein the confirmation is based on a selection at the terminal of the medical device from the subset of the plurality of medical devices.

Clause 8. The medical information system of any one of Clauses 2 through 7, wherein the one or more computing devices are further configured to: receive for each respective medical device of the plurality of medical devices, a clinician assigned to the respective medical device; receive, from the terminal after presenting the plurality of medical devices, a selected clinician; determine a subset of the plurality of medical devices based determining that the selected clinician is assigned to the subset of the plurality of medical devices; and present, for display at the terminal, the subset of the plurality of medical devices as being ready to receive automated programming, wherein the confirmation is based on a selection at the terminal of the medical device from the subset of the plurality of medical devices.

Clause 9. An infusion device, comprising: a pump controller comprising a processor; wherein the pump controller is configured to: determine that the infusion device is in an operational state to program the infusion device to perform a therapy; activate a user interface element configured to transmit, via a communication network, a first message indicating that the infusion device is ready to receive automated programming; receive an activation of the user interface element; responsive to receiving the activation, send the first message to a medical information system, indicating that the infusion device is ready to receive automated programming; receive, from the medical information system after sending the first message, an automated programming message; and configure, responsive to receiving the automated programming message, the infusion device according to predetermined operational parameters associated with the received automated programming message.

Clause 10. The infusion device of Clause 9, wherein configuring the infusion device comprises programming the infusion device to perform the therapy based on drug library information stored in a drug library deployed to the infusion device.

Clause 11. The infusion device of Clause 9 or Clause 10, further comprising: a touchscreen, wherein activating the user interface element comprises displaying a virtual button on the touchscreen.

Clause 12. A computer-implemented method, comprising, by a medical information system: receiving, from a medical device remote from the medical information system, a first message indicating that the medical device is ready to receive automated programming; receiving, from a terminal remote from the medical device, a request to automatically program an unidentified medical device according to an automated programming message, the request not identifying the medical device; presenting for display at the terminal, based on the first message and responsive to the request, the medical device as being ready to receive automated programming; receiving, from the terminal after presenting the medical device as being ready to receive automated programming, a confirmation that the automated programming message is for the medical device; and automatically causing, responsive to receiving the confirmation, the automated programming message to be transmitted to the medical device, the automated programming message causing the medical device to be configured according to predetermined operational parameters.

Clause 13. The computer-implemented method of Clause 12, further comprising: receiving a plurality of messages indicating that a plurality of medical devices are ready to receive automated programming; presenting, for display at the terminal, the plurality of medical devices as being ready to receive automated programming, wherein the confirmation is based on a selection at the terminal of the medical device from the plurality of medical devices.

Clause 14. The computer-implemented method of Clause 13, wherein receiving the plurality of messages comprises receiving a plurality of channel designations corresponding to a plurality of medical device modules coupled to a patient care device; wherein presenting the plurality of medical devices as being ready to receive automated programming comprises presenting the plurality of channel designations for the patient care device, wherein the confirmation comprises a selected channel designation of the plurality of channel designations; and causing the automated programming commend to be transmitted to the patient care device, the automated programming message instructing the patient care device to configure a medical device module corresponding to the selected channel designation according the predetermined operational parameters.

Clause 15. The computer-implemented method of Clause 14, wherein receiving a plurality of messages indicating that the plurality of medical devices are ready to receive automated programming comprises: receiving an identifier of the patient care device; and determining the plurality of channel designations based on indexing a database based on the identifier of the patient care device.

Clause 16. The computer-implemented method of Clause 14 or Clause 15, further comprising: receiving an infusion status associated with each of the plurality of channel designations; and presenting, for display at the terminal, each infusion status together with each associated channel designation.

Clause 17. The computer-implemented method of any one of Clauses 14 through 16, wherein the automated programming message causing the medical device to be configured according to predetermined operational parameters comprises the automated programming message causing the medical device to be configured by drug library information stored in a drug library deployed to the medical device.

Clause 18. The computer-implemented method of any one of Clauses 13 through 17, further comprising: receiving, for each respective medical device of the plurality of medical devices, a care area associated with the respective medical device; receiving, from the terminal after presenting the plurality of medical devices, a selected care area; determining a subset of the plurality of medical devices based matching the selected care area with a care area of one or more of the respective medical devices; and presenting, for display at the terminal, the subset of the plurality of medical devices as being ready to receive automated programming, wherein the confirmation is based on a selection at the terminal of the medical device from the subset of the plurality of medical devices.

Clause 19. The computer-implemented method of any one of Clauses 13 through 18, further comprising: receiving, for each respective medical device of the plurality of medical devices, a clinician assigned to the respective medical device; receiving, from the terminal after presenting the plurality of medical devices, a selected clinician; determining a subset of the plurality of medical devices based determining that the selected clinician is assigned to the subset of the plurality of medical devices; and presenting, for display at the terminal, the subset of the plurality of medical devices as being ready to receive automated programming, wherein the confirmation is based on a selection at the terminal of the medical device from the subset of the plurality of medical devices.

Clause 20. A non-transitory computer readable medium comprising instructions that, when executed by a computing system, cause the computing system to perform a method according to any one of Clauses 12 through 19.

### Further Considerations

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” when used to describe a relationship between two or more elements encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A medical information system, comprising:
one or more computing devices configured to:
receive, from medical devices remote from the medical information system, a plurality of messages indicating that the medical devices are ready to receive automated programming;
list the medical devices in a database as being ready to receive automated programming;
receive, from a terminal remote from the medical devices, a request to automatically program an unidentified medical device according to an automated programming message, the request not identifying any medical device;
responsive to the request, search a database for and identify all active medical devices in the database that are indicated as being ready to receive automated programming;
present for display at the terminal, responsive to the request and based on the search, a list of the medical devices that are ready to receive automated programming;
receive, from the terminal after presenting the list of medical devices that are ready to receive automated programming, a selection of a medical device from the list for the request; and
automatically cause, responsive to receiving the selection, the automated programming message to be transmitted to the medical device, the automated programming message causing the medical device to be configured according to predetermined operational parameters.

2. The medical information system of claim 1,
wherein receiving the plurality of messages comprises receiving a plurality of channel designations corresponding to a plurality of medical device modules coupled to a patient care device;
wherein presenting the plurality of medical devices as being ready to receive automated programming comprises presenting the plurality of channel designations for the patient care device;
wherein the confirmation comprises a selected channel designation of the plurality of channel designations; and
wherein the automated programming message instructs the patient care device to configure a medical device module corresponding to the selected channel designation according the predetermined operational parameters.

3. The medical information system of claim 2, wherein receiving a plurality of messages indicating that the plurality of medical devices are ready to receive automated programming comprises:
receiving an identifier of the patient care device; and
determining the plurality of channel designations based on indexing a database based on the identifier of the patient care device.

4. The medical information system of claim 2, or wherein the one or more computing devices are further configured to:
receive an infusion status associated with each of the plurality of channel designations; and
present, for display at the terminal, each infusion status together with each associated channel designation.

5. The medical information system of claim 2, wherein the automated programming message causing the medical device to be configured according to predetermined operational parameters comprises the automated programming message causing the medical device to be configured by drug library information stored in a drug library deployed to the medical device.

6. The medical information system of claim 1, wherein the one or more computing devices are further configured to:
receive, for each respective medical device of the plurality of medical devices, a care area associated with the respective medical device;
receive, from the terminal after presenting the plurality of medical devices, a selected care area;
determine a subset of the plurality of medical devices based matching the selected care area with a care area of one or more of the respective medical devices; and
present, for display at the terminal, the subset of the plurality of medical devices as being ready to receive automated programming,
wherein the confirmation is based on a selection at the terminal of the medical device from the subset of the plurality of medical devices.

7. The medical information system of claim 1, wherein the one or more computing devices are further configured to:
receive for each respective medical device of the plurality of medical devices, a clinician assigned to the respective medical device;
receive, from the terminal after presenting the plurality of medical devices, a selected clinician;
determine a subset of the plurality of medical devices based determining that the selected clinician is assigned to the subset of the plurality of medical devices; and
present, for display at the terminal, the subset of the plurality of medical devices as being ready to receive automated programming,
wherein the confirmation is based on a selection at the terminal of the medical device from the subset of the plurality of medical devices.

8. An infusion device, comprising:
a touchscreen; and
a pump controller comprising a processor;
wherein the pump controller is configured to:
determine that the infusion device is in an operational state to program the infusion device to perform a therapy;
displaying a virtual button on the touchscreen, the virtual button configured to cause a transmission, via a communication network, of a first message indicating that the infusion device is ready to receive automated programming;
receive an activation of the user interface element;
responsive to receiving the activation, send the first message to a medical information system, indicating that the infusion device is ready to receive automated programming;
receive, from the medical information system after sending the first message, an automated programming message; and
configure, responsive to receiving the automated programming message, the infusion device according to predetermined operational parameters associated with the received automated programming message.

9. The infusion device of claim 8, wherein configuring the infusion device comprises programming the infusion device to perform the therapy based on drug library information stored in a drug library deployed to the infusion device.

10. A computer-implemented method, comprising, by a medical information system:
receiving, from medical devices remote from the medical information system, a plurality of messages indicating that the medical device are ready to receive automated programming;
listing the medical devices in a database as being ready to receive automated programming;
receiving, from a terminal remote from the medical devices, a request to automatically program an unidentified medical device according to an automated programming message, the request not identifying any medical device;
responsive to the request, searching a database for and identifying all active medical devices in the database that are indicated as being ready to receive automated programming;
presenting for display at the terminal, responsive to the request and based on the searching, a list of the medical device that are ready to receive automated programming;
receiving, from the terminal after presenting the list of medical devices that are ready to receive automated programming, a selection of a medical device from the list for the request; and
automatically causing, responsive to receiving the selection, the automated programming message to be transmitted to the medical device, the automated programming message causing the medical device to be configured according to predetermined operational parameters.

11. The computer-implemented method of claim 10,
wherein receiving the plurality of messages comprises receiving a plurality of channel designations corresponding to a plurality of medical device modules coupled to a patient care device;
wherein presenting the plurality of medical devices as being ready to receive automated programming comprises presenting the plurality of channel designations for the patient care device, wherein the confirmation comprises a selected channel designation of the plurality of channel designations; and
causing the automated programming commend to be transmitted to the patient care device, the automated programming message instructing the patient care device to configure a medical device module corresponding to the selected channel designation according the predetermined operational parameters.

12. The computer-implemented method of claim 11,
wherein receiving a plurality of messages indicating that the plurality of medical devices are ready to receive automated programming comprises:
receiving an identifier of the patient care device; and
determining the plurality of channel designations based on indexing a database based on the identifier of the patient care device.

13. The computer-implemented method of claim 11, further comprising:
receiving an infusion status associated with each of the plurality of channel designations; and
presenting, for display at the terminal, each infusion status together with each associated channel designation.

14. The computer-implemented method of claim 11, wherein the automated programming message causing the medical device to be configured according to predetermined operational parameters comprises the automated programming message causing the medical device to be configured by drug library information stored in a drug library deployed to the medical device.

15. The computer-implemented method of claim 10, further comprising:
receiving, for each respective medical device of the plurality of medical devices, a care area associated with the respective medical device;
receiving, from the terminal after presenting the plurality of medical devices, a selected care area;
determining a subset of the plurality of medical devices based matching the selected care area with a care area of one or more of the respective medical devices; and
presenting, for display at the terminal, the subset of the plurality of medical devices as being ready to receive automated programming,
wherein the confirmation is based on a selection at the terminal of the medical device from the subset of the plurality of medical devices.

16. The computer-implemented method of claim 10, further comprising:
receiving, for each respective medical device of the plurality of medical devices, a clinician assigned to the respective medical device;
receiving, from the terminal after presenting the plurality of medical devices, a selected clinician;
determining a subset of the plurality of medical devices based determining that the selected clinician is assigned to the subset of the plurality of medical devices; and
presenting, for display at the terminal, the subset of the plurality of medical devices as being ready to receive automated programming,
wherein the confirmation is based on a selection at the terminal of the medical device from the subset of the plurality of medical devices.

17. A non-transitory computer readable medium comprising instructions that, when executed by a computing system, cause the computing system to perform a method according to claim 10.
